(19) United States
(12) Patent Application Publication
(10) Pub. No.: US 2015/0086646 A1
(43) Pub. Date: Mar. 26, 2015

(54) ANESTHETIC NEUTRALIZATION METHODS

(71) Applicant: Instigo Biotech Ltd., Tel-Aviv (IL)

(72) Inventors: Sagi Polani, Tel-Aviv (IL); Asher Polani, Tel-Aviv (IL); Roee Atlas, Givatayim (IL)

(21) Appl. No.: 14/391,009

(22) PCT Filed: Apr. 7, 2013
(86) PCT No.: PCT/IB2013/052770

§ 371 Date: Oct. 7, 2014

Related U.S. Application Data
(60) Provisional application No. 61/621,528, filed on Apr. 8, 2012.
(60) Provisional application No. 61/748,453, filed on Jan. 3, 2013.

(51) Int. Cl. A61K 33/40

(52) U.S. Cl. CPC A61K 33/40
USPC 424/616; 424/613; 514/236.2

(57) ABSTRACT

Embodiments of the invention relates to decreasing effects of local and/or regional anesthesia by administering to a patient in need thereof an agent selected from the group of agents consisting of reactive nitrogen species (RNS), reactive oxygen species (ROS) and/or an agent which induces formation of RNS and/or ROS. Exemplary agents which may be used include peroxynitrite and hydrogen peroxide.

### RELATED APPLICATIONS

The present application claims the benefit under 35 U.S.C. 119(e) of U.S. Provisional Application 61/621,528 filed on Apr. 8, 2012, and U.S. Provisional Application 61/748,453 filed on Jan. 3, 2013 the disclosures of which are incorporated herein by reference.

### TECHNICAL FIELD

Embodiments of the invention relate to methods of decreasing local/regional anesthetic effects.

### BACKGROUND

Anesthesia is a loss of sensation and/or insensitivity to pain in a human or animal, typically induced by an agent called an anesthetic. Anesthetics are typically used to allow surgeons to perform surgery, diagnosis and other medical procedures which would be painful without anesthesia.

Anesthesia is typically divided into three types, local, regional and general anesthesia. Local and regional anesthesia involve inhibiting sensation from an area or organ of a body by blocking nerve transmission to and from the area or organ. Local anesthesia typically refers to anesthesia of a small area or organ. For example, local anesthesia is typically induced by administering a local anesthetic to the gums before performing a dental procedure such as removing a tooth. Regional anesthesia typically refers to blocking sensation from a general region of a body by blocking nerve impulses from the general region of a body to the spinal cord and/or from the spinal cord to the brain. An example of regional anesthesia is epidural anesthesia, which may be used to ease pain during childbirth.

Local and regional anesthetics can be administered alone or in combination with a vasoconstricting agent, an agent which reduces blood flow by narrowing blood vessels, thereby prolonging the local effects of the anesthetic by preventing systemic absorption and clearance of the locally administered anesthetic. Various vasoconstricting agents may be used with local or regional anesthetics. A common combination used to induce local anesthesia is lidocaine with epinephrine.

General anesthesia involves inducing a temporary state of unconsciousness in a patient in which the patient is sedated, insensitive to pain and the patient's skeletal muscles are relaxed. General anesthesia is typically used for prolonged and invasive surgeries and is typically administered by specialists with expertise in anesthesia to ensure patient safety throughout the anesthetic treatment.

It is often advantageous for patient comfort and recovery that the effects of an anesthetic in producing numbness and lack of feeling be alleviated relatively rapidly upon completion of a medical procedure in which the anesthetic was used. Moreover, in cases where a vasoconstrictor is used in combination with a local anesthetic, a prolonged numbness may be evident for up to several hours after surgery. Such numbness may be inconvenient or even restrict the patient from returning to normal daily routines. For example, numbness of the tongue, cheeks and lips following dental surgery may complicate eating, drinking and even talking. In veterinary practice, an animal may damage a locally/regionally anesthetized surgical site, for example, by chewing sutures, because post-operative numbness prevents the animal from feeling pain associated with damaging the surgical site. In such cases the return to normal sensation is advantageous in preventing self-inflicted harm and potential suffering of the animal.

In addition to inconveniences associated with numbness induced by local and regional anesthetics, prolonged anesthetic effects may even hinder medical procedures. In reconstructive dental surgery, a surgeon anesthetizes the surgical site before beginning surgery and is careful not to damage regional nerves. In order to test for nerve damage, it is beneficial that the local/regional anesthetic effect wears off so that the surgeon can confirm that there is no need for further surgical procedures to fix potential nerve damage, or to ensure that no nerve damage has occurred prior to continuing with the medical procedure, (e.g. dental implantations). In this sense, if bilateral surgery is indicated, the surgeon is limited to performing surgery only on one side until full sensation is regained prior to continuing surgery to the contralateral side in order to assess for any potential nerve damage. Moreover, when determining the source of lameness in veterinary orthopedics, it is beneficial that the anesthetic effect wears off after administration of regional nerve-blocks in order to localize the source of lameness in the animal as quickly as possible. Prolonged anesthetic effects can increase the time it takes for a veterinarian to diagnose the source of lameness.

### SUMMARY

An aspect of embodiments of the invention relates to decreasing effects of local and/or regional anesthesia by administering to a patient in need thereof an agent selected from the group of agents consisting of reactive nitrogen species (RNS) reactive oxygen species (ROS) and an agent which induces formation of RNS and/or ROS.

RNS are highly active nitrogen-containing molecules, ions or radicals. Examples of RNS include nitric oxide, nitrite ion, nitrogen dioxide, dinitrogen trioxide, nitrate ion, nitrogen trioxide, nitrosoperoxycarbonate, nitrogen dioxide radical, nitrogen trioxide radical, nitrosoperoxycarbonate radical, peroxynitrite and nitric oxide radical.

ROS are highly active oxygen-containing molecules, ions or radicals, typically having an unpaired oxygen atom. ROS are produced in the body of organisms and are typically associated with oxidative damage within organisms. Examples of ROS include superoxide, hydrogen peroxide, hydroxyl radical and carbonate radical. Collectively, the group of molecules and ions consisting of RNS and ROS are known as ROS/RNS.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to a figure attached hereto that is listed following this paragraph.

FIG. 1 shows a flow diagram depicting methods of treating a patient in need of local/regional anesthesia according to embodiments of the invention; and

FIG. 2 shows a bar graph depicting effects of ROS and RNS in decreasing effects of regional anesthesia by increasing response to stimuli time in a rat model at various time points following administration.

### DETAILED DESCRIPTION

Studies have shown that in acute inflammatory tissues, effects of local anesthetics are decreased when compared to effects of local anesthetics in healthy tissue. Many hypotheses have been proposed to explain the phenomenon including reduction in the pH of inflammatory tissue, which decreases effects of the local/regional anesthetic, or increased vascularity/vasodilation in inflamed tissue leading to removal of local/regional anesthetics from the tissue (Takakura et al., 2005, Ueno et al., 2008a). To date, methods comprising introduction of ROS/RNS to tissue to decrease the effects of local and/or regional anesthesia after the administration of local and/or regional anesthetics have not been suggested. The inventors suggest that use of ROS/RNS in accordance with embodiments of the invention has beneficial effects in reducing effects of local and/or regional anesthesia.

Without being bound by theory, it is suggested that decreasing effects of local and/or regional anesthesia are achieved through one or more of the activities of ROS/RNS including: membrane lipid peroxidation, permeation of biological membranes, direct interaction with local anesthetics and inhibition of their membrane fluidizing effects, potassium(K)-ATPase channel activation via ATP depletion/PARS activation and membrane hyperpolarization (essentially promoting vasodilation), hyperpolarization of smooth muscles by K-ATPase sensitive channel activation, activation of guanylate cyclase and in turn elevation of cGMP which induces vascular smooth muscle relaxation, and interference with endothelial cells' calcium entry by calcium channel depletion essentially inhibiting calcium induced vasoconstriction (Ueno et al., 2008b, Gunaydin and Demiryurek, 2001; Radi et al., 1991). In addition, it is suggested that RNS and in particular peroxynitrite, are capable of deactivating dopamine, norepinephrine, and epinephrine, namely catecholamines, thus decreasing their vasoconstrictive effects (Daveu et al., 1997; Kuo et al., 2003; Takakura et al., 2003a; Takakura et al., 2003b). Similar effects have been documented for treatment of epinephrine with ROS, e.g. H₂O₂ and its degradation products, superoxide and hydroxyl radical (Babior and Kipnes, 1976; Macarthur et al., 2000).

In the following detailed description, new methods of treatment using ROS/RNS and/or agents which produce ROS/RNS in a patient will be described in detail.

### Example 1

### Treatment of a Patient in Need of Local and/or Regional Anesthetic

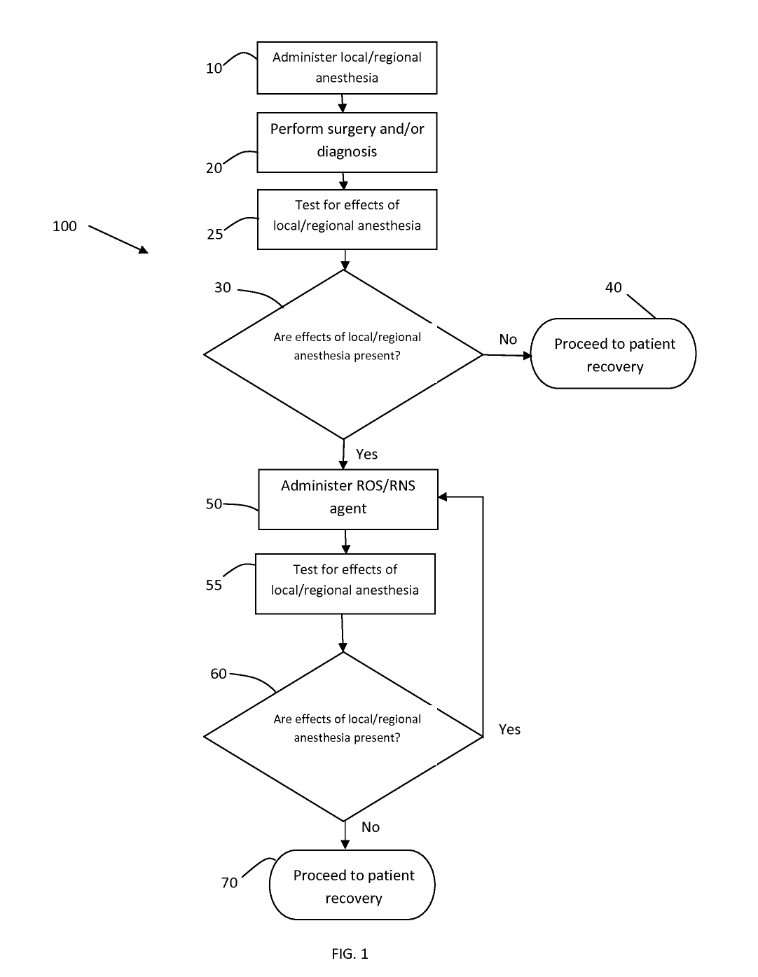

FIG. 1 shows a flow-diagram depicting a method 100 for administration of a local/regional anesthetic to a patient according to an embodiment of the invention. In an embodiment of the invention, method 100 is performed on a patient in need of a diagnostic procedure and/or a surgical procedure which is associated with pain or discomfort in the patient. The patient is a human or animal patient. In embodiments of the invention, the patient is a mammalian patient. Method 100 comprises administering local and/or regional anesthesia in a section 10 and in a section 20 performing surgery and/or diagnosis. In a section 25 at a time following surgery or diagnosis when it is considered advantageous for effects of the local/regional anesthetia on the patient to have worn off, a procedure is carried out to determine if such effects are present. In a decision section 30, if effects of local/regional anesthesia are determined not to be present method 100 proceeds to a section 40. In section 40 the patient proceeds to patient recovery. If unwanted effects of local/regional anesthesia are determined to be present, in a section 50 ROS/RNS or an agent which increases production of ROS/RNS is administered to the patient. After administration of ROS/RNS or an ROS/RNS inducing agent in a section 55 the patient is tested for unwanted local/regional anesthetic effects. In a decision section 60, if effects of local/regional anesthesia are determined not to be present method 100 proceeds to a section 70. In section 70, the patient proceeds to patient recovery. If effects of local/regional anesthesia are determined to be present in decision section 60, ROS/RNS or an agent which increases production of ROS/RNS is administered to the patient as in section 50.

Examples of such procedures according to section 20 in which method 100 can be performed according to embodiments of the invention include surgeries and procedures associated with local and/or regional anesthesia selected from the group consisting of dental, oral, oropharyngeal, nasopharyngeal, pharyngeal, otolaryngeal mandibular, maxillofacial, orthopedic, reconstructive, soft-tissue, plastic surgery, dermal, endocrine, trauma-related, oncological, gastro-intestinal, colorectal, vascular, neurological, gynecological, urological, ophtalmological, cardiothoracic or obstetric procedures and/or surgeries.

The procedure in which method 100 can be performed according to embodiments of the invention include elective surgeries, emergency surgeries, exploratory surgeries, cosmetic surgeries, transplant surgeries, laparoscopic surgeries, laser surgeries and microsurgeries.

In embodiments of the invention, the procedure is a veterinary diagnostic procedure to determine the source of lameness in an animal, for example in a horse. In such a procedure, at least one nerve (e.g. nerve-block) and/or joint of the animal is anesthetized in order to determine if lameness stems from pain within the innervated region and/or joint and/or additional anatomical structures that communicate with the joint. If lameness persists despite administration of a local anesthetic, another joint is anesthetized in order to diagnose the source of lameness.

Local/regional anesthetics are administered in section 10 in an amount sufficient to cause loss of pain sensation in a region associated with the surgery and/or diagnosis according to section 20. In an embodiment of the invention, the patient's response to pain or sensation in the region may be tested, for example, by pinching the location of surgery in order to confirm that the local/regional anesthetic administration was successful in blocking pain sensation. Multiple administrations may be required to maintain anesthesia to the region throughout the course of the surgery or diagnosis according to section 20.

In an embodiment of the invention, local/regional anesthetic is administered according to section 10 through the gingival, topical, corneal, intradermal, transdermal, subcutaneous, percutaneous, trans-mucosal, intramuscular, intra-articular or epidural routes.

In an embodiment, local/regional anesthetic is administered according to section 10 to section a nerve selected from the group consisting of brachial plexus, lumbosacral plexus, coccygeal plexus, trigeminal, facial, auriculopalpebral, maxillary, mandibular, mental, mental, buccal, zygomatic, dorsal scapular, suprascapular, subscapular, musculocutaneus, axillary, median, radial, ulnar, volar, palmar/plantar, palmar/plantar digital, palmar-metacarpal/plantar-metatarsal, iliohypogastric, posterior, gluteual, ilioinguinal, genitofemoral, femoral, sciatic, obturator, tibial, saphenous, peroneal, pudendal and cutaneous nerves and their relative branches. In an embodiment, local/regional anesthesia is administered according to section 10 to perform epidural anesthesia, paravertebral anesthesia, obstetrical analgesia, surgical analgesia, intravenous regional anesthesia or intra-articular anesthesia.

In an embodiment of the invention, anesthesia administered in section 10 is an anesthetic selected from the group consisting of benzocaine, chloroprocaine, cocaine, cyclomethycaine, dimethocaine/larocaine, piperocaine, propoxycaine, procaine/novocaine, proparacaine, tetracaine/amethocaine, articaine, bupivacaine, cinchocaine/dibucaine, etidocaine, levobupivacaine, lidocaine/lignocaine/xylocaine/esracaine, mepivacaine/carbocaine, prilocaine, ropivacaine, trimecaine, oxybuprocaine, saxitoxin, tetrodotoxin, menthol and eugenol. In an embodiment the anesthetic is administered in combination with a vasoconstrictor. In an embodiment the vasoconstrictor is epinephrine/adrenaline.

Upon termination of surgery and/or diagnosis according to section 20, the effects of local/regional anesthesia are tested in accordance with section 25. In an embodiment, the tested effect of local/regional anesthesia is pain sensation. In an embodiment, the patient is tested for pain sensation by a mildly painful contact such as a pinch or a prod optionally accompanied by a verbal questioning of the patient, in the case of a human patient, to determine if the patient felt pain or by a visual analysis to determine if the patient responds to the mildly painful contact, for example, by twitching. If the effects of anesthesia are no longer present, patient may continue to recovery as in section 40. If the effects of anesthesia are present, the patient is administered ROS/RNS in accordance with section 50.

In an embodiment of the invention, ROS/RNS and/or an agent which causes the production of ROS/RNS administered in accordance with section 50 comprises peroxynitrite or a salt thereof.

Peroxynitrite may be administered in the form of sodium peroxynitrite, also known as peroxynitrous acid, sodium salt and ONO2Na, from manufacturers including Cayman Chemical Company, Ann Arbor Mich., USA. Basic solutions of peroxynitrite may be formed and administered according to section 50.

In an embodiment of the invention, ROS/RNS or an agent which causes the production of ROS/RNS in accordance with section 50 may be formulated with at least one pharmaceutically acceptable excipient e.g. sodium hydroxide, glycine, sodium chloride and hydrochloride acid. In an embodiment, the formulation is in the form of a gel, solution for injection, topical solution, ointment, suspension or topical cream.

In an embodiment of the invention, ROS/RNS, and in particular peroxynitrite may be formulated with dimethyl sulfoxide (DMSO) which was found to have protective mechanism against potentially harmful side effects when administered locally (such as inflammatory damage) associated with peroxynitrite (Jia et al., 2010). In an embodiment the concentration of DMSO is about 0.005-0.5%.

In accordance with an embodiment of the invention, ROS/RNS or an agent which causes the production of ROS/RNS is administered according to section 50 to the same region of the body of the patient as the anesthetic agent is administered in accordance with section 10.

In an embodiment of the invention, ROS/RNS or an agent which causes the production of ROS/RNS is administered according to section 50 through the gingival, topical, corneal, intradermal, transdermal, subcutaneous, percutaneous, trans-mucosal, intramuscular, intra-articular and epidural routes.

In an embodiment of the invention, ROS/RNS or an agent which causes the production of ROS/RNS is administered according to section 50 in combination with a vasodilating agent. Exemplary vasodilating agents may include nitric oxide or nitroglycerine. In an embodiment of the invention, ROS/RNS or an agent which causes the production of ROS/RNS is administered according to section 50 in combination with an agent which may modify catecholamines. Examples of such agents include nitrite and nitrate. An alpha-adrenergic blocking agent such as phentolamine and salts thereof may also be used in combination with ROS/RNS or an agent which causes the production of ROS/RNS.

An exemplary agent according to an embodiment of the invention that can cause production of ROS/RNS is a peroxynitrite donor. An example of a peroxynitrite donor is 3-morpholinosynonimine-N-ethyl-carbamine, also known as SIN-1 (Takakura et al., 2005). Upon contact with molecular oxygen in vivo, SIN-1 reacts to form NO and superoxide, which together form peroxynitrite.

According to an embodiment of the invention ROS/RNS or an agent which causes the production of ROS/RNS is administered to the patient in accordance with section 50 to achieve a relatively low concentration of ROS/RNS in affected tissue, in order to prevent systemic absorption of ROS/RNS which may be associated with harmful side-effects. In an embodiment, the tissue concentration or ROS/RNS is between 0.01 and 100 millimolar (mM). In an embodiment, a volume of solution between 0.1 and 15 milliliters (ml) of solution having an ROS/RNS concentration of between 0.01 and 100 mM is administered to obtain a tissue concentration between about 0.01 and 100 mM.

In certain surgical procedures, a medical practitioner performs a procedure which may unintentionally cause nerve damage, for example, by applying pressure to a nerve, thereby causing pain in a patient. An example of such a surgical procedure is a dental implantation. If the procedure is performed under local or regional anesthesia, it may be difficult for the medical practitioner to determine if he has unintentionally caused nerve damage as the effects of local/regional anesthetic prevent the patient from feeling pain sensation for extended periods (up to several hours) after the surgery. In such a situation, unintentional nerve damage may only be recognized by the patient after the patient has completed the surgery and returned home from the hospital/clinic. In addition to the discomfort caused to the patient, such nerve damage can be time-consuming and costly to repair once the patient has already left the hospital or clinic. According to an embodiment of the invention a medical practitioner can reduce the effects of local/regional anesthesia in order to ensure that no potential iatrogenic nerve damage was elicited during surgery. If the medical practitioner determines that nerve damage has been caused, he or she may re-administer local/regional anesthetics in order to allow further surgical intervention to correct the nerve damage.

According to an embodiment of the invention, the medical practitioner can determine if the surgery was successful by asking the patient regarding pain after surgery and after determining reduced effects of local/regional anesthesia according to section 55. Since effects of local/regional anesthesia have been reduced, patient may be able to sense pain or sensation indicative of nerve damage, if nerve damage was sustained during the surgery. According to an embodiment of the invention, the medical practitioner can determine if the surgery was successful by testing the patient for pain after surgery, and if the patient experiences significantly more pain than normal after verifying that local/regional anesthetic effects have been reduced, this may be indicative of nerve damage elicited during the surgery.

If the surgery was determined not to be successful, for example, the patient determined that excess pain was present, the medical practitioner may repeat the surgery or parts thereof in order to eliminate the source of pain. In an embodiment, upon determining that the surgery was not successful, the medical practitioner may return to administer a local/regional anesthetic agent in accordance with section 10.

If the surgery was determined to be successful, for example the patient was able to sense pain and no potential nerve damage was indicated, the patient may proceed to recovery as in section 70.

Methods according to embodiments of the invention can be time-saving and money-saving for medical practitioners and patients as patients obviate the need to wait extended periods of time to recover from unwanted effects of local/regional anesthesia.

### Example 2A

### Determination of Source of Lameness in Horses

Lameness in animals such as horses can be difficult to treat and diagnose, as horses do not readily communicate with humans to address the source of their pain. Diagnostic local anesthesia (“nerve or joint block”) is a technique used for determining the source of lameness in animals. When performing diagnostic nerve blocks, local anesthetic is infused either around a nerve or injected into a joint or other synovial structure, for example a tendon sheath or bursa. If the lameness disappears or improves markedly following administration of anesthesia, a veterinarian will have localized the source of lameness and will be able to treat the lameness accordingly. However, if lameness does not disappear or improve following administration of local anesthesic, the veterinarian may choose to apply local anesthetic to a second nerve/synovial structure/joint in a second region or joint. In order for the veterinarian's diagnosis to be accurate, he may want to wait until the anesthetic effect in the first nerve/synovial structure/joint dissipates before applying local anesthetic to a second nerve/synovial structure/joint in/around the second region or joint. Such a procedure may be time consuming because of the long lasting effect of local anesthetics and may require nerve blocks in multiple nerves/synovial structures/joints before successful diagnosis.

Nerve blocks in combination with administration of ROS/RNS according to embodiments of the invention may be used to assist in diagnosis of the source of lameness in animals, thereby decreasing the time it takes for a veterinarian to successfully diagnose the source of lameness.

In an embodiment of the invention, a veterinarian performs a nerve block to a first nerve/synovial structure/joint suspected of being associated with the source of lameness. If lameness does not disappear or improve, the veterinarian then administers ROS/RNS or an agent which produces ROS/RNS to the first site of injection. The veterinarian then performs a nerve block to the second site suspected of being associated with the source of lameness. The veterinarian may then administer ROS/RNS or an agent which produces ROS/RNS to the second site if the nerve block does not improve the lameness. This process may be continued until the source of lameness is determined by the veterinarian.

### Example 2B

### Orthopedic Procedures

Joint luxation, in which a bone becomes displaced from its normal position within a joint may occur as a result of trauma such as falls or impacts. In case of a joint luxation, a patient may suffer from extreme pain. Because the process of repairing the luxation by moving the bone into place is painful, local/regional anesthesia may be used.

According to an embodiment of the invention, a patient suffering from a joint luxation is treated with local or regional anesthesia, and then an orthopedic intervention at the joint is performed in order to repair and restore the luxated joint to normal positioning. In order to test if the joint is properly restored, the patient is then administered an effective amount of ROS/RNS to the affected area to test if the patient feels pain. A lack of pain or reduced pain relative to the pain experienced during luxation may indicate successful repositioning of the luxated joint. On the other hand, excess/unconventional pain relative to the pain experienced during luxation may indicate potential iatrogenic damage to the joint, thereby indicating a need to intervene once again in order to return/repair the potential iatrogenic damage.

### Example 2C

### Obstetric Procedures

During vaginal parturition (childbirth), a common way to alleviate pain associated with parturition involves administration of regional anesthetics via the epidural route. However, a disadvantage of using this treatment is muscular weakness during and for a period of time of up to four hours after the birth.

According to an embodiment of the invention, after alleviation of pain of childbirth, a patient may be administered an effective amount of ROS/RNS via the epidural route to decrease the regional anesthetic effect and reduce unnecessary numbness and/or muscle weakness associated with the regional anesthesia.

### Example 2D

### Dental Procedures

In order to maintain dental health, a dentist may perform painful dental procedures such as filling cavities or extracting teeth using local anesthetics. Despite the widespread use of local anesthetics in dentistry, it may be associated with negative effects. For example, following a dental procedure in which local/regional anesthesia is used, a patient's mouth or parts thereof may remain numb for hours. A patient may unknowingly bite his or her tongue, cheek or lip, thereby causing bleeding and/or a wound. In addition, local/regional anesthetic agents administered to certain parts of the mouth or jaw in dental or mandibular or maxillofacial surgery may also impact the mental, buccal, zygomatic, mandibular, maxillary, trigeminal or facial nerves, causing a phenomenon known as “nerve palsy” resulting in weakness, drooping or immobility of muscles such as the facial muscles.

According to an embodiment of the invention, after performing a dental, mandibular or maxillofacial procedure, a patient may be administered an effective amount of ROS/RNS to counteract the local anesthetic effect and reduce unnecessary numbness and/or muscle weakness associated with local anesthesia. According to an embodiment of the invention, the dental procedure is a tooth filling, a root canal, an implantation, an extraction, reconstruction or arthrocentesis.

### Example 3

### Testing of ROS/RNS compounds in rats for decreasing regional anesthetic

effects

An animal model was performed to test ROS/RNS dosages and effects on sensory anesthesia following administration of regional anesthesia, as follows. Male rats (SD Rat) weighing between 300-350 grams, were supplied by Harlan, Israel, and were randomly assigned to experimental groups. Rats were provided with sterile rodent diet with free access to drinking water.

In the animal model, a testing chamber (Plantar test 3730 apparatus, made by Ugo Basile) was used in which the rats were placed on a surface which is heated and increases heat gradually. A measurement of time from placement of the rat's hind limb on the hot surface to lifting the hind limb was recorded. Increased time to lifting the hind limb indicated that regional anesthesia was blocking nerve sensation and response to heat.

All rats were habituated to the testing facilities for five days before the model, and then introduced into the testing chamber two days prior to the testing of regional anesthesia in order to habituate rats to the apparatus. One day prior to regional anesthetic administration, baseline body weight measurements and baseline paw thermal sensitivity were recorded.

Rats were randomly divided into 7 groups having 4 rats in each group. Group 1 was not administered anesthesia, but was administered saline solution as a vehicle at the time of regional anesthetic administration to groups 2-7. Groups 2 through 7 were administered lidocaine HCl 2%/epinephrine 1:100,000 ready to use solution (hereinafter, “anesthetic,”) 7 minutes before administering test items. Regional anesthetic was injected around the sciatic and saphenous nerves at a volume of 150 microliters at each site of the rat's left hind limb.

Injection around the saphenous nerve was performed via local injection to/next to the adductor canal which runs on the medial aspect of the thigh between the vastus medialis and the sartorius muscles, about 2-3 cm below the inguinal canal. Injection around the sciatic nerve was performed via local injection to the sciatic notch, positioned 1 mm from the femoral shaft between the greater trochanter and ischial tuberosity pointing toward the ischium.

Five minutes after injection of regional anesthetic, an assessment of the nerve blocks was performed by pinching the rat's paw to determine if the regional anesthetics were properly administered.

Seven minutes after regional anesthetic administration, test agents were administered to the same loci, in a volume of 150 microliters at each locus. Groups 1 and 2 were administered a vehicle buffer as test agents. Group 3 was administered 0.1 mM hydrogen peroxide, formulated in saline solution. Groups 4, 5, 6, and 7 were administered an RNS agent, namely peroxynitrite (PON), at concentrations of 40, 10, 1 and 0.1 mM respectively.

PON which was administered to rats was prepared by commercially available sodium peroxynitrite in NaCl and NaOH each at a concentration of 0.3 molar, available from Cayman Chemical. The commercially available PON was diluted to the desired concentrations for injection, by forming a suspension in 0.3M NaCl, 0.1M glycine having a pH of 10.5.

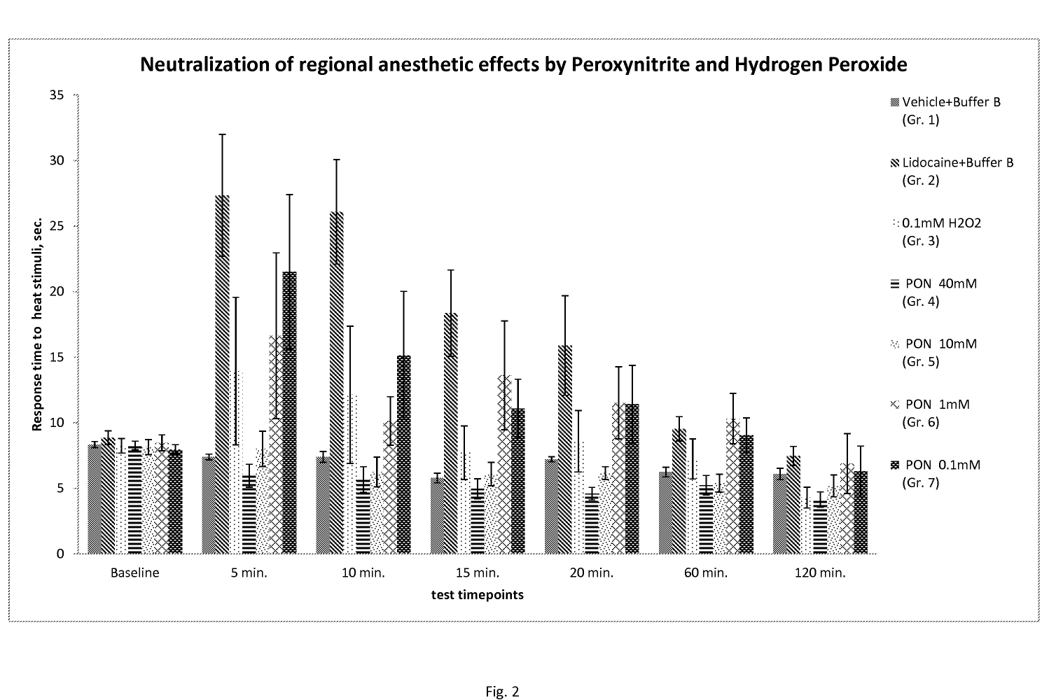

All rats were tested for paw thermal sensitivity at 5, 10, 15, 20, 60 and 120 minutes after administration of the test items using the testing chamber. The results of average time before lifting the treated limb in each rat for each time point was calculated and is depicted in FIG. 2.

As can be seen in the bar graph in FIG. 2, baseline measurements of response time for all groups were similar, indicating similar paw thermal sensitivity before administration of regional anesthesia. In locally anesthetized rats which were treated with the buffer alone as a test agent (Group 2), response time was relatively long at the 5, 10, and 15 minute time points (20-35 seconds), but gradually decreased over time, as the effects of the regional anesthesia wore off. As early as 5 minutes after administration of PON in groups 4 and 5, response time was decreased to levels comparable to those of the control group 1 (4-6 seconds), in which no anesthetic was administered, indicating that in these groups, regional anesthetic neutralization was achieved in a short time. Group 6 also showed improved response time at the 5 and 10 minute time points relative to group 2 thus manifesting a dose-dependent response.

The effect of the ROS agent, H₂O₂, was also seen as evident by decreased response time in group 3 rats at the 5, 10 and 15 minute time points relative to group 2. Although an effect of H₂O₂ was seen, this effect was not statistically significant, possibly due to the relatively small number of rats used.

This model shows that that RNS and in particular peroxynitrite and ROS and in particular H₂O₂ are effective in quickly eliminating effects of regional anesthesia.

There is further provided in accordance with an embodiment of the invention a method for decreasing the effects of a local or regional anesthetic agent in a patient comprising administering to the patient an ROS/RNS or an agent which produces ROS/RNS.

There is further provided in accordance with an embodiment of the invention a pharmaceutical composition comprising an ROS/RNS or an agent which produces ROS/RNS for decreasing the effects of a local or regional anesthetic agent in a patient.

There is further provided in accordance with an embodiment of the invention use of ROS/RNS or an agent which produces ROS/RNS for the manufacture of a medicament for decreasing the effects of a local or regional anesthetic agent in a patient. Optionally, the patient was administered an amount of an anesthetic agent to reduce sensation of pain, prior to the administration of the ROS/RNS or agent which produces ROS/RNS. Optionally, a surgical or diagnostic procedure was performed on the patient prior to the administration of the ROS/RNS or agent which produces ROS/RNS. Optionally, the ROS/RNS is peroxynitrite or hydrogen peroxide. Optionally, the ROS/RNS is nitric oxide, nitrite ion, nitrogen dioxide, dinitrogen trioxide, nitrate ion, nitrogen trioxide, nitrosoperoxycarbonate, nitrogen dioxide radical, nitrogen trioxide radical, nitrosoperoxycarbonate radical, nitric oxide radical, superoxide, hydroxyl radical and carbonate radical. Optionally, the patient was administered a vasoconstricting agent in conjunction with the anesthetic agent. Optionally, the anesthetic agent is administered locally or regionally. Optionally, the anesthetic agent administered is in the form of local/regional anesthesia, synovial anesthesia, a nerve block, joint block or epidural. Optionally, the diagnostic procedure is associated with localization and assessment of the source of pain and/or nerve damage in a human/animal. Optionally, the surgical procedure is a dental, oral, oropharyngeal, nasopharyngeal, pharyngeal, otolaryngeal, mandibular, maxillofacial, orthopedic, reconstructive, soft-tissue, plastic surgery, dermal, endocrine, trauma-related, oncological, gastro-intestinal, colorectal, vascular, neurological, gynecological, urological, ophthalmological, cardiothoracic or obstetric procedure. Optionally, the obstetric procedure is parturition. Optionally, dental procedure is a tooth filling, a root canal, an implantation, an extraction, reconstruction, plastic surgery or arthrocentesis. Optionally, the orthopedic procedure is the repair or replacement or restoration of a bone into a joint. Optionally, effects of local/regional anesthesia are decreased upon administration of a volume of solution between 0.1 and 15 milliliters (ml) of solution having an ROS/RNS concentration of between 0.01 and 100 mM. Optionally, the ROS/RNS or an agent which produces ROS/RNS and the anesthetic agent are administered via the same route. Optionally, the ROS/RNS or an agent which produces ROS/RNS is administered in conjunction with a second pharmaceutical agent. Optionally, the second pharmaceutical agent is selected from the group consisting of: a vasodilating agent, an agent which modifies catecholamines and an alpha-adrenergic blocking agent. Optionally, the anesthetic agent is selected from the group consisting of: benzocaine, chloroprocaine, cocaine, cyclomethycaine, dimethocaine/larocaine, piperocaine, propoxycaine, procaine/novocaine, proparacaine, tetracaine/amethocaine, articaine, bupivacaine, cinchocaine/dibucaine, etidocaine, levobupivacaine, lidocaine/lignocaine/xylocaine/esracaine, mepivacaine/carbocaine, prilocaine, ropivacaine, trimecaine, oxybuprocaine, saxitoxin, tetrodotoxin, menthol and eugenol, and a pharmaceutically acceptable salt of any of the anesthetic agents. Optionally, the vasoconstricting agent is epinephrine/adrenaline.

In the description and claims of the present application, each of the verbs, “comprise,” “include” and “have,” and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

### WORKS CITED

Babior B M and Kipnes R S. 1976. Oxidation of Epinephrine by a Cell-free System From Human Granulocytes. Blood, 47(3): 461-471.
Daveu C, Servy C, Dendane M, Marin P and Ducrosq C. 1997. Oxidation and Nitration of Catecholamines by Nitrogen Oxides Derived from Nitric Oxide. Nitric Oxide, 1(3): 234-243.
Gunaydin B and Demiryurek A T. 2001. Interaction of Lidocaine with reactive oxygen and nitrogen species. European Journal of Anesthesiology, 18: 816-822.
Jia Z, Zhu H, Li Y and Misra H P. 2010. Potent inhibition of peroxynitrite-induced DNA strand breakage and hydroxyl radical formation by dimethyl sulfoxide at very low concentrations. Experimental Biology and Medicine, 235(5): 614-622.
Kuo W N, Kocis J M and Guruvadoo L K. 2003. Modification of tyrosine and catecholamines by peroxynitrite, nitrite and nitrate. Frontiers in Bioscience, 8: A139-A142.
Macarthur H, Westfall T C, Rilley D P, Misko T P and Salvemini D. 2000. Inactivation of catecholamines by superoxide gives new insights on the pathogenesis of septic shock. PNAS, 97(17): 9753-9758.
Radi R, Beckman J S, Bush K M and Freeman B A. 1991. Peroxynitrite-induced membrane lipid peroxidation: The cytotoxic potential of superoxide and nitric oxide. Archives of Biochemistry and Biophysics, 288: 481-487.
Takakura K, Xiaohong W, Takeuchi K, Yasuda Y and Fukuda S. 2003. Deactivation of norepinephrine by Peroxynitrite as a new pathogenesis in the hypotension of septic shock. Anesthesiology, 98(4): 928-934.
Takakura K, Xiaohong W, Takeuchi K and Fukuda S. 2003. Peroxynitrite decreases dopamine's vasoconstrictive activity. Anesthesia and Analgesia, 97(5): 1492-1496.
Takakura K, Mizogami M, Ono Y, Ooshima K and Muramatsu I. 2005. Decrease of the inhibitory effect of Lidocaine on trigeminal nerve response by the inflammatory oxidant Lidocaine. Canadian Journal of Anesthesia, 52(4): 439-440.
Ueno T, Tsuchiya H, Mizogami M and Takakura K. 2008. Local anesthetic failure associated with inflammation: verification of the acidosis mechanism and the hypothetic participation of inflammatory peroxynitrite. Journal of Inflammation Research, 1: 41-48.
Ueno T, Mizogami M, Takakura K and Tsuchiya H. 2008. Membrane effect of Lidocaine is inhibited by interaction with peroxynitrite. Journal of Anesthesia, 22: 96-99.

1. A method for decreasing the effects of a local or regional anesthetic agent in a patient comprising administering to a patient a ROS/RNS or an agent which produces ROS/RNS.

2. The method according to claim 1 in which the patient was administered an amount of an anesthetic agent to reduce sensation of pain, prior to the administration of the ROS/RNS or agent which produces ROS/RNS.

3. The method according to claim 2 in which a surgical or diagnostic procedure was performed on the patient prior to the administration of the ROS/RNS or agent which produces ROS/RNS.

4. The method according to claim 1 wherein the ROS/RNS is peroxynitrite.

5. The method according to claim 1 wherein the ROS/RNS or agent which produces ROS/RNS is nitric oxide, nitrite ion, nitrogen dioxide, dinitrogen trioxide, nitrate ion, nitrogen trioxide, nitrosoperoxycarbonate, nitrogen dioxide radical, nitrogen trioxide radical, nitrosoperoxycarbonate radical, nitric oxide radical, superoxide, hydroxyl radical and carbonate radical.

6. The method according to claim 1 wherein the patient is administered a vasoconstricting agent in conjunction with the anesthetic agent.

7. The method according to claim 1 wherein the local/regional anesthetic agent is administered to the patient.

8. The method according to claim 7 wherein the local or regional anesthetic agent is administered in the form of a nerve block, a joint block, synovial anesthesia, or epidural anesthesia.

9. The method according to claim 3 wherein the diagnostic procedure is associated with localization and assessment of the source of pain and/or nerve damage in a human/animal.

10. The method according to claim 3 wherein the surgical procedure is a dental, oral, oropharyngeal, nasopharyngeal, pharyngeal, otolaryngeal, mandibular, maxillofacial, orthopedic, reconstructive, soft-tissue, plastic surgery, dermal, endocrine, trauma-related, oncological, gastro-intestinal, colorectal, vascular, neurological, gynecological, urological, ophtalmological, cardiothoracic or obstetric procedure.

11. The method according to claim 10 wherein the obstetric procedure is parturition.

12. The method according to claim 10 wherein the dental procedure is a tooth filling, a root canal, an implantation, an extraction, reconstruction, plastic surgery or arthrocentesis.

13. The method according to claim 10 wherein the orthopedic procedure is the repair or replacement or restoration of a bone into a joint.

14. The method according to claim 1 wherein effects of local/or regional anesthesia are decreased upon administration of a volume of solution between 0.1 and 15 milliliters (ml) of solution having an ROS/RNS concentration of between 0.01 and 100 mM.

15. The method according to claim 2 wherein the ROS/RNS or an agent which produces ROS/RNS and the anesthetic agent are administered via the same route.

16. The method according to claim 1 wherein the ROS/RNS or an agent which produces ROS/RNS is administered in conjunction with a second pharmaceutical agent.

17. The method according to claim 16 wherein the second pharmaceutical agent is selected from the group consisting of: a vasodilating agent, an agent which modifies catecholamines and/or an alpha-adrenergic blocking agent.

18. The method according to claim 2 wherein the anesthetic agent is selected from the group consisting of: benzocaine, chloroprocaine, cocaine, cyclomethycaine, dimethocaine/larocaine, piperocaine, propoxycaine, procaine/novocaine, proparacaine, tetracaine/amethocaine, articaine, bupivacaine, cinchocaine/dibucaine, etidocaine, levobupivacaine, lidocaine/lignocaine/xylocaine/esracaine, mepivacaine/carbocaine, prilocaine, ropivacaine, trimecaine, oxybuprocaine, saxitoxin, tetrodotoxin, menthol and eugenol, and a pharmaceutically acceptable salt of any of the anesthetic agents.

19. The method according to claim 6 wherein the vasoconstricting agent is epinephrine/adrenaline.

20. The method according to claim 1 wherein the ROS/RNS is hydrogen peroxide.
